(19) United States
(12) Patent Application Publication
(10) Pub. No.: US 2011/0293712 A1
(43) Pub. Date: Dec. 1, 2011

(54) COAXIALLY ENCAPSULATED CELL SUSPENSIONS AND MICROTISSUES

(75) Inventors: Ralph Kurt, Eindhoven (NL); Emiel Peeters, Eindhoven (NL); Roel Penterman, Eindhoven (NL); Dirk J. Broer, Geldrop (NL); David Halter, Eindhoven (NL)

(73) Assignee: KONINKLIJKE PHILIPS ELECTRONICS N.V., EINDHOVEN (NL)

(21) Appl. No.: 13/132,534

(22) PCT Filed: Nov. 26, 2009
(86) PCT No.: PCT/IB2009/055349

§ 371 Date: Aug. 22, 2011

(30) Foreign Application Priority Data
Dec. 2, 2008 (EP) 08170418.1

(51) Int. Cl. A61K 9/48; A61K 35/12; C12M 1/00

(52) U.S. Cl. 424/451; 435/283.1; 424/93.7

(57) ABSTRACT

The present invention provides for the coaxial encapsulation of a plurality of cells in a single elongated compartment. By this encapsulation, the cells are protected by at least one layer of separation material and kept in close contact, which leads to a better vitality of the encapsulated cells and consequently results in higher chances to form microtissue. Methods and devices for the production of such encapsulated cell compartments are disclosed as well as medical uses of such compartments in cell, tissue therapy and tissue engineering.

### FIELD OF THE INVENTION

The present invention relates to the field of regenerative medicine, particularly to cell therapy and tissue engineering and provides devices and methods for the coaxial encapsulation of cell suspensions and/or the generation of microtissue. Furthermore, coaxially encapsulated cell suspensions, microtissues and tissues, as well as their medical uses are disclosed.

### BACKGROUND OF THE INVENTION

Regenerative medicine is a new upcoming discipline within the field of medical sciences. There are numerous methods and approaches used in regenerative medicine.

Cell therapies are based on the delivery of cells, particularly stem cells, to a damaged tissue area in order to restore the tissue function.

In tissue engineering, tissue is grown outside the body utilizing scaffolds and cells. The engineered tissue is subsequently implanted in a patient in order to replace damaged or lost tissue.

The handling and growing of the cells is a crucial factor in these applications. In general, cells are first harvested from the appropriate cell source, i.e. from the patient (“autologous”) or from a donor (alleogenic) and subsequently subjected to several different steps until they are finally introduced again into the patient to replace or restore damaged tissue. Despite the tremendous progress in cell therapies and tissue engineering over the last few years, basic and essential steps, e.g. cell handling, cell (in)growth in scaffolds, cell differentiation, cell delivery and cell retainment are still problematic and need further improvement before regenerative medicine becomes clinically relevant.

Particularly, three main problems persist in the field of cell therapy and tissue engineering. Firstly, cells injected in a patient need to stay located in the area of the damaged tissue, for example, the site of a lesion and kept viable. The basic principle of (stem-) cell therapies is to introduce (stem-) cells in an area of injury or disease. The newly introduced (stem-) cells have the ability to grow, divide and differentiate, and can repopulate the damaged site with newly formed tissue that has the same or a similar structure and function as the native tissue. The main concern in cell therapies under development lies in the fact that for many therapies large excesses of cells are needed. Often, only 1% of the injected cells are delivered and/or retained in the area of interest and survives the first week. Cells are expensive and difficult to obtain in large quantities (time consuming). Therefore, means are required to contain such injected cells and prevent them from dislodging from the site of injection while allowing these cells to grow, replicate and differentiate.

Secondly, the provision of heterogeneous (micro)tissues, e.g. vascular tissue or nerve tissue is still a major challenge.

Thirdly, tissue engineered in vitro can currently only be of a rather limited thickness (in the range of about 100 μm). This is due to the fact that when growing these tissues no vascular system is present which allows supplying all of the cells of the engineered tissue with, for example, nutrients and oxygen. Thus, especially the innermost cells of a tissue engineered by current means tend to die off.

### SUMMARY OF THE INVENTION

It is the object of the present invention to provide means to overcome these problems. Particularly, it is an object of the invention to provide means to allow for the encapsulation of a plurality of cells into a single coaxial and elongated compartment, wherein the encapsulated cells can communicate with each other in order to increase their vitality and thus the chance to form microtissue as cell-cell contacts are formed easier. Thus, it is one object of the present invention to provide a method for the generation of coaxial and elongated encapsulated cell suspensions comprising a plurality of cells with the possibility to communicate with each other.

Furthermore, the present invention provides means to grow tissue of a thickness of more than 100 μm from such elongated compartments comprising a plurality of cells. This is due to the fact that such elongated compartments can be cultured into fibrous microtissues which can, in a further step, be arranged or stacked in order to form tissues of increased thickness. Such tissues can also be heterogeneous tissues, i.e. they can comprise different cell types.

In one aspect of the invention a nozzle 8 for coaxial extrusion is provided, comprising at least two coaxial cylinders, wherein each cylinder contains a cell suspension 1, a solution comprising a cell signaling molecule or a separation material 3, wherein cylinders containing a cell suspension or a solution comprising a signaling molecule are separated by a cylinder containing a separation material, and wherein the outermost cylinder contains a separation material.

In a further aspect of the invention an extruder comprising the nozzle according to the invention is provided.

In an even further aspect of the invention the use of the nozzle or the extruder according to the invention for the generation of at least one compartment containing a cell suspension encapsulated by a layer of separation material is provided.

In a further aspect of the invention a method for the encapsulation of a cell suspension and/or the generation of microtissue is provided, the method comprising: coaxially extruding at least one cell suspension, at least one separation material and optionally at least one solution comprising a cell signaling molecule, wherein cell suspensions or solutions comprising a signaling molecule are separated by separation material and wherein the resulting outermost layer consist of separation material.

In a further aspect of the invention an encapsulated cell suspension or microtissue obtainable by the methods according to the invention is provided.

In a further aspect of the invention the encapsulated cell suspension or microtissue of the invention for the use as a medicament is provided.

In another aspect of the invention a method for the generation of tissue is provided comprising the step of stacking at least two layers of microtissue according to the invention.

Finally, a tissue comprising at least two stacked layers of microtissue according to the invention or obtainable by the method of the invention is provided.

Additional details, features, characteristics and advantages of the object of the invention are disclosed in the dependent claims, the figures and the detailed description.

### BRIEF DESCRIPTION OF THE DRAWINGS

The figures show, in an exemplary fashion, preferred embodiments of the present invention. In particular,

FIG. 1 shows a cross section of a nozzle 8 according to the present invention comprising two coaxial cylinders. The central cylinder contains a cell suspension 1 comprising a plurality of cells 2. The outer cylinder contains a separation material 3. Furthermore a radiation source 9 and a closure or sealing of the extruded product 7 are shown.

FIG. 2 FIGS. 2a and 2b show a vertical and horizontal cross section, respectively through the product extruded by the nozzle displayed in FIG. 1 (encapsulated cell suspension) and depicts a cell suspension 1 comprising a plurality of cells 2, the separation material 3 forming the outer protective layer and a closure or sealing 7 of the extruded product.

FIG. 3 FIGS. 3a and 3b show the extruded product of FIG. 2 at a later time;

preferably after culturing of the extruded product. As can be seen, the multiple individual cells depicted in FIG. 2 have turned into a microtissue, wherein the cells are attached to the layer(s) of separation material.

FIG. 4 shows a cross section of a nozzle 8 according to the present invention comprising four coaxial cylinders. The central cylinder contains a cell suspension 1 comprising a plurality of cells 2 of a first type, followed by a surrounding cylinder containing a separation material 3 and a further surrounding cylinder containing a cell suspension 4 comprising a plurality of cells 5 of a second type. The outer cylinder contains a separation material 6 than can be the same or different from the other separation material 3. Furthermore two sources of radiation 9 and a closure or sealing of the extruded product (7) are shown.

FIG. 5 FIGS. 5a and 5b show a vertical and horizontal cross section, respectively through the product extruded by the nozzle displayed in FIG. 4 and depict the two cell suspensions 1 and 4 comprising a plurality of cells 2 and 5, the separation material 3 and 6 and a closure or sealing 7 of the extruded product.

FIG. 6 FIGS. 6a and 6b show the extruded product of FIG. 5 at a later time; preferably after culturing of the extruded product. As can be seen, the multiple individual cells depicted in FIG. 5 have turned into a microtissue, wherein the cells are attached to the layer(s) of separation material. In case different cell types are used as cells 2 and 5 a heterogeneous microtissue is thus provided.

### DETAILED DESCRIPTION OF EMBODIMENTS

The present invention provides means that allow for the coaxial encapsulation of a cell solution comprising a plurality of cells in a single elongated compartment or multiple connected elongated compartments. By this encapsulation, the cells are protected by at least one layer of separation material and kept in close contact. This contact between the cells surprisingly leads to a better vitality of the encapsulated cells and consequently results in higher chances to form microtissue.

Furthermore, the encapsulated cell suspensions of the invention and/or microtissues developed therefrom can be used in cell therapy and/or in vivo microtissue delivery applications as the encapsulated cells and/or microtissue will be retained at the site of injection/insertion into the patient. In conventional methods, cell suspensions are simply injected into, e.g., the site of a lesion and the cells are prone to dislodge from that site instead of differentiating into functional tissue replacing the affected former tissue.

Thus, the present inventions provides for means to more reliably deliver cells or microtissue to a patient in need thereof, wherein the cells are kept viable and chances of tissue formation are increased. Furthermore, the cells and/or developed tissue(s) are more likely to remain at the position(s) within the patient's body, e.g. a site of a lesion, into which they have been injected/inserted. Thus, chances of formation of functional replacement tissue are drastically increased.

If the separating material(s) used according to the invention is/are biodegradable the injected/inserted cells and/or microtissues will—upon insertion/injection into the patient and after a given time—no longer be encapsulated by the separation material as the separating material(s) will have degraded. Thus, the inserted/injected cells and/or microtissue(s) will develop into functional tissue that seamlessly integrates with the patient's body.

Furthermore, the present invention also provides for the encapsulation of more than one type of cells into coaxially nested/stacked compartments. By this means heterologous tissues may be generated that are of high value in microtissue delivery applications as, for example, functional vascular tissue or nervous tissue may be provided.

In addition, the present invention provides for the encapsulation of at least one cell suspension into a first compartment adjacent to a solution comprising a cell signaling molecule that is encapsulated in a second compartment that is coaxially surrounding the first compartment or coaxially surrounded by said first compartment. By this means, cell signaling molecules that are important for, e.g., cell differentiation can reliably be supplied to the cells.

The present invention furthermore provides for the generation of tissue with a thickness of more than about 100 μm. Producing such tissues, one is generally faced with the problem of maintaining the cells located within the tissue as no vascular system is present yet.

The present invention provides means to generate such tissue, wherein the microtissues of the present invention are stacked on top of each other in order to yield a tissue with a thickness of ≧100 μm, preferably ≧1 cm and even more preferably ≦2 cm. The stacking of the microtissues of the present invention allows nutrients to be delivered to all cells of the developing tissue even though no vascular system is present. In a specific embodiment the tissue has a thickness of ≧100 μm and ≦5 cm. In another specific embodiment the tissue has a thickness of ≧300 μm and ≦2 cm. In another specific embodiment the tissue has a thickness of ≧500 μm and ≦1 cm.

In the following, the present invention is demonstrated by means of preferred embodiments and examples, which by no means should be understood as to limit the scope of the invention.

### DEFINITIONS

The term “nozzle” as used herein, refers to any device that is suitable to be employed in the coaxial extrusion methods according to the invention. Extrusion is a process used to create objects of a fixed cross-sectional profile, wherein a material, for example the separation material, is pushed or drawn through a die of the desired cross-section. The minimal dimension of the nozzle should be such that cells with typical sizes of 5-50 μm can be encapsulated.

In one embodiment the inner diameter of the nozzle is ≧10 μm and ≦1 cm. In another embodiment the inner diameter is ≧250 μm and ≦750 μm. In another embodiment the inner diameter is ≧350 μm and ≦550 μm.

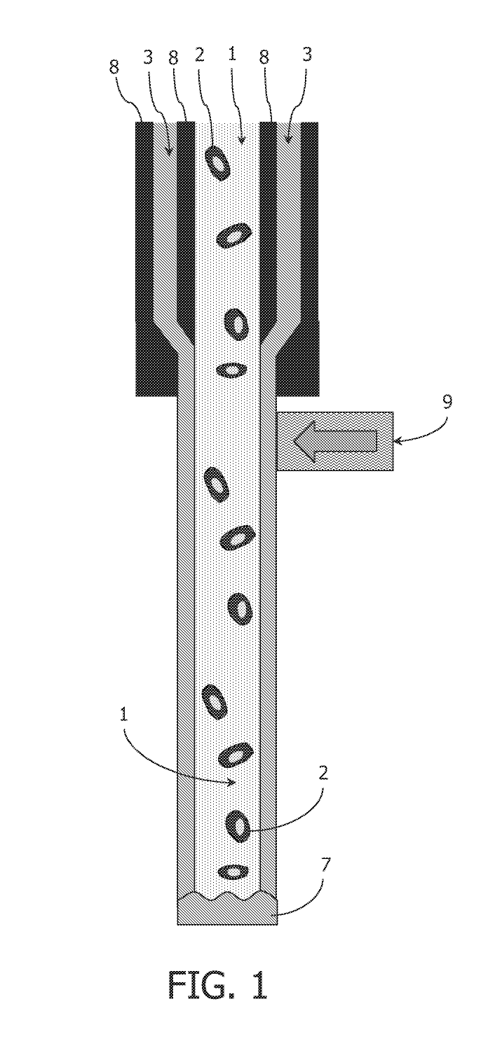

The term “cylinder” as used herein, refers to the coaxially stacked tubes forming a nozzle. However, for reasons of simplicity, the term “cylinder” also refers to the space formed between two of such tubes. As an example reference is made to FIG. 1. Here, the nozzle 8 is made up of two coaxially stacked tubes (displayed in black) which result in the formation of two spaces. The first and innermost space (displayed dotted) that is formed contains the cell suspension 1, while the second space (displayed hatched) coaxially surrounding the first space contains the separation material 3. Thus, for example, the term “outermost cylinder” may refer to either the outermost tube forming the nozzle (displayed in black) or it may refer to the hatched outermost space formed between the innermost tube and the outermost tube and that contains the separation material 3.

In one embodiment the distance between two consecutive coaxial tubes making up a cylinder containing separation material is ≧100 nm and ≦1 mm, ≧300 nm and ≦800 μm, ≧500 nm and ≦500 μm, or ≧1 μm and ≦200 μm. Thus, the thickness of a layer of separation material extruded from such cylinder is ≧100 nm and ≦1 mm, ≧300 nm and ≦800 μm, ≧500 nm and ≦500 μm, or ≧1 μm and ≦200 μm.

The minimal distance between two consecutive coaxial tubes making up a cylinder containing a cell suspension is governed by the diameter of the extruded cells. In further embodiments the distance between two consecutive coaxial tubes making up such a cylinder containing a cell suspension is ≧5 μm, preferably ≧5 μm and ≦5 cm, ≧10 μm and ≦2 cm, ≧20 μm and ≦1 cm, ≧30 μm and ≦500 μm, ≧40 μm and ≦400 μm, ≧50 μm and ≦300 μum, ≧100 μm and ≦500 μm, or ≧200 μm and ≦400 μm. These distances also apply for cylinders containing a solution comprising a cell signaling molecule. Thus, the thickness of a layer of a solution comprising a cell signaling molecule or of a layer of a cell suspension extruded from such cylinder is ≧5 μm, preferably ≧5 μm and ≦5 cm, ≧10 μm and ≦2 cm, ≧20 μm and ≦1 cm, ≧30 μm and ≦500 μm, ≧40 μm and ≦400 μm, ≧50 μm and ≦300 μm, ≧100 gm and ≦500 μm, or ≧200 μm and ≦400 μm.

In a further embodiment of the invention the wall thickness of the cylinder is at ≧20 μm and ≦500 μm, preferably at least ≧30 μm and ≦100 μm.

In a further embodiment, the length of each of the coaxial cylinders is ≧500 μm and ≦10 cm, preferably ≧1 mm and ≦5 cm, ≧3 mm and ≦5 cm, ≧3 mm and ≦3 cm, or ≧5 mm and ≦3 cm.

An “extruder” as used herein refers to any apparatus that is suitable to push or draw material, e.g. separation material, a cell suspension, and/or a solution comprising a cell signaling molecule through a nozzle according to the invention. In specific embodiments the extruder consists of syringe pumps using standard syringes, wherein the syringes are connected to the nozzle by, e.g., small tubes.

In particular embodiments the flow rate of the separation material, a cell suspension, and/or a solution comprising a cell signaling molecule is ≧0 m/min and ≦10 m/min, ≧0 m/min and ≦8 m/min, ≧0 m/min and ≦6 m/min, ≧0 m/min and ≦4 m/min, ≧0 m/min and ≦2 m/min, ≧0 m/min and ≦1.5 m/min, ≧0 m/min and ≦1 m/min, or ≧0 m/min and ≦0.5 m/min.

The term “cell suspension” as used herein refers to a plurality of cells suspended in any suitable fluid that allows for the maintenance, propagation, differentiation or growth of the cells. In one embodiment the fluid is a cell nutrition medium. A cell nutrition medium can, inter alia, be a culturing or a growth medium and the exact ingredients will depend on the type of cells the cell suspension contains. An example for a cell nutrition medium is Dulbecco's Modified Eagle's Medium (DMEM, Gibco) culture medium, supplemented with 10% fetal calf serum (FCS), 1% penicillin/streptomycin and 1% Glutamax (Invitrogen).

In specific embodiments of the present invention the cell suspension has a concentration of ≧1 and ≦10⁹ cells per ml, in a particular embodiment the cell suspension has a concentration of ≧1000 and ≦10⁸, more preferably ≧10⁵ and ≦10⁷ cells per ml. Most preferably, the cell suspension has a concentration of about 2×10⁵ cells/ml.

In further specific embodiments of the present invention the cells are selected from the group consisting of stem cells, progenitor cells, fully-differentiated cells, neural cells, glial cells, Schwann cells, oligodendrocytes, adipose-derived stem cells (ASCs), mesenchymal stem cells (MSCs), Pancreatic progenitor cells, feeder cells, endothelial progenitor cells (EPC), pluripontent stem cells, somatic stem cells, myeloid stem cells and/or lymphoid stem cells.

Human ASCs have been shown to differentiate into bone, cartilage, fat, and muscle, while ASCs from rats have been converted to neurons.

MSCs are multipotent stem cells that can differentiate into a variety of tissues and can be isolated from umbilical cord blood, Wharton's jelly, placenta, adipose tissue, lung, and bone marrow. MSCs are attractive for clinical therapy due to their ability to differentiate, provide trophic support, and modulate innate immune response. EPC are very important for research on fracture and wounds healing.

The cells in a single cell suspension can be cells of a single cell type or more cell types. In a further embodiment the cells are cultured cells.

Furthermore, it may be provided any of the following additives may be added to the cell suspension or solution comprising a cell signalling molecule:

(i) agents which enhance the bio compatibility of the extruded product;

(ii) biodegradable and/or biologically safe material;

(iii) labels or markers; and/or

(iv) growth factors;

(v) antibiotics.

The term “microtissue” as used herein refers to at least one layer of aggregated cells that is generated when the cells in the extruded cell solution settle on the layer(s) of separation material (see, for example FIGS. 3a and 6a). Thus, the term microtissue refers to a confluent layer of cells, wherein contact between the cells is established.

The term “cell signaling molecule” as used herein refers to any molecule that has an effect on maintenance, propagation, differentiation, de-differentiation or growth of a given cell. Preferred embodiments of such cell signaling molecules are growth factors, cytokines and/or chemokines

In specific embodiments of the present invention the cell signaling molecule is at least one molecule selected from the group consisting of G-CSF, GM-CSF, SCF, IL-3, IL-6, IGF-1, fibroblast growth factor (FGF), basic FGF (bFGF), transforming growth factor β1 (TGF-β1), activin-A, bone morphogenic protein 4(BMP-4), hepatocyte growth factor (HGF), epidermal growth factor (EGF), β nerve growth factor (β-NGF), retinoic acid, FGF-1, FGF-2, Thrombopoietin (TPO) and/or Erythropoietin (EPO).

In a specific embodiment, the cell signaling molecule is insuline-like growth factor 1 (IGF-1) that has been shown to promote myocyte proliferation.

In a further specific embodiment, the cell signaling molecule is a neurotrophin. Neurotrophins are a family of proteins that induce the survival, development and function of neurons. They belong to a class of growth factors, secreted proteins, which are capable of signaling particular cells to survive, differentiate, or grow. Growth factors such as neurotrophins that promote the survival of neurons are known as neurotrophic factors. Neurotrophic factors are secreted by target tissue and act by preventing the associated neuron from initiating programmed cell death—thus allowing the neurons to survive. Neurotrophins also induce differentiation of progenitor cells, to form neurons.

In a further specific embodiment, the cell signaling molecule is the hepatocyte growth factor/scatter factor (HGF/SF) that is a paracrine cellular growth, motility and morphogenic factor. It is secreted by mesenchymal cells and targets and acts primarily upon epithelial cells and endothelial cells, but also acts on haemopoietic progenitor cells. It has been shown to have a major role in embryonic organ development, in adult organ regeneration and in wound healing. Hepatocyte growth factor regulates cell growth, cell motility, and morphogenesis by activating a tyrosine kinase signaling cascade after binding to the proto-oncogenic c-Met receptor. Hepatocyte growth factor is secreted by mesenchymal cells and acts as a multi-functional cytokine on cells of mainly epithelial origin. Its ability to stimulate mitogenesis, cell motility, and matrix invasion gives it a central role in angiogenesis, tumorogenesis, and tissue regeneration.

In a further specific embodiment, the cell signaling molecule is Growth differentiation factor 9 (GDF9). Growth factors synthesized by ovarian somatic cells directly affect oocyte growth and function. GDF9 is expressed in oocytes and is thought to be required for ovarian folliculogenesis. GDF9 is a member of the transforming growth factor-beta (TGFβ) superfamily. GDF9 plays an important role in the development of primary follicles in the ovary. It has a critical role in granulosa cell and theca cell growth, as well as in differentiation and maturation of the oocyte. GDF9 has been connected to differences in ovulation rate and in premature cessation of ovary function, therefore has a significant role in fertility.

In one embodiment of the invention the cytokine is fibroblast growth factor (FGF)-20. FGF-20 is a member of the fibroblast growth factor (FGF) family whose family members possess broad mitogenic and cell survival activities, and are involved in a variety of biological processes including embryonic development cell growth, morphogenesis, tissue repair, tumor growth and invasion. FGF-20 was shown to be expressed in normal brain, particularly the cerebellum and to be able to enhance the survival of midbrain dopaminergic neurons in vitro. FGF-20 was further shown to have potent effects to generate large numbers of dopaminergic neurons derived from hESCs.

The solution of cell signaling molecules will initially be separated from the cell suspension by means of the separation material surrounding the cell suspension. In one embodiment the separation material is biodegradable and thus will lead to a contacting of the cells within the cell suspension and the signaling molecules over time. In another embodiment the separation material is permeable for the cell signaling molecules (and not for the cells) and thus, depending on the permeability coefficient, allows for a controlled diffusion of the cell signaling molecules into the compartment comprising the cell suspension.

The term “separation material” as used herein refers to any kind of material that is suitable to separate two liquid mediums. In one embodiment the term “separate” means that a mixing of the two said liquid mediums is prevented. Preferably, the separation material is suitable to separate a cell suspension from a solution comprising a cell signaling molecule and/or to separate a cell suspension from another cell suspension. A separation material according to the invention can be a gel, for example a hydrogel, a fluid that auto-gelates, or a polymerizing substance.

Preferred separation materials are, inter alia, hydrogels based on polymer networks derived from monomers such as PEGacrylate, pNiPAAm, PAAm, PHEMA, PHEA in combination with crosslinker molecules such as PEGDA, PEGDMA, DEGDA, DEGDMA, TEGDA, TEGDMA, biodegradable crosslinker molecules di(meth)acrylate materials containing oligolactide segments, oligo glycolide segments, oligocaprolactone segments and other oligoester segments. Alternatively alginate or peptide amphiphile nanofiber based hydrogels can be used as well as porous polymeric system (membranes) obtained by Polymerization induced phase separation (PIPS) or temperature responsive hydrogels such as pNiPAAm and other temperature responsive hydrogels known in the art.

In specific embodiments, such porous polymeric systems/membranes are obtainable by photo-polymerization induced phase separation of a mixture comprising a cross-linking monomer and a single porogenic solvent, wherein the solvent concentration is ≧40 and ≦80 wt %, preferred ≧42 and ≦70 wt %, more preferred ≧45 and ≦60 wt %, most preferred about 50 wt %.

In further particular embodiments the membrane is obtainable by photo-polymerization induced phase separation of said mixture, wherein the cross-linking monomer is tetraethyleneglycoldimethylacrylate. In more preferred embodiments the single porogenic solvent is either polypropyleneglycol or tripropyleneglycol.

In another preferred embodiment said membrane is produced, wherein the mixture comprises glycidylmethacrylate and/or acrylic acid as further reactive monomer.

Porogenic solvents are those solvents which are suitable for forming pores and/or displacing the polymer chains during polymerization. The characteristics and use of such solvents in the formation of macroreticular or macroporous resins are described in U.S. Pat. No. 4,224,415, which is included herein by reference. A porogenic solvent is one which dissolves the monomer mixture being (co-)polymerized but which does not dissolve the (co-)polymer. In addition, the porogenic solvents must be inert to the polymerization conditions, i.e., neither interfere with or enter into the polymerization.

According to an embodiment of the present invention, the material mixture of which the porous membrane is made of is a polymeric material comprising a poly(meth)acrylic material.

According to another embodiment of the present invention, the porous membrane comprises a poly(meth)acrylic material made by the polymerization of at least one (meth)acrylic monomer and at least one polyfunctional (meth)acrylic monomer.

According to another embodiment of the present invention, the (meth)acrylic monomer is chosen from the group comprising (meth)acrylamide, (meth)acrylic acid, hydroxyethyl (meth)acrylate, ethoxyethoxyethyl(meth)acrylate, hydroxyethylmeth(meth)acrylate, isobornyl(meth)acrylate, isobornyl meth(meth)acrylate or mixtures thereof.

According to a further embodiment of the present invention, the polyfunctional (meth)acrylic monomer is a bis-(meth)acryl and/or a tri-(meth)acryl and/or a tetra-(meth)acryl and/or a penta-(meth)acryl monomer.

According to another embodiment of the present invention, the polyfunctional (meth)acrylic monomer is chosen out of the group comprising bis(meth)acrylamide, tripropyleneglycol di(meth)acrylates, pentaerythritol tri(meth)acrylate, polyethyleneglycoldi(meth)acrylate, ethoxylated bisphenol-A-di(meth)acrylate or mixtures thereof.

According to an embodiment of the present invention, the crosslink density in the poly(meth)acrylic material is 0.05 and 1, preferably 0.1 and 0.8, more preferably 0.4 and 0.5.

In one embodiment the separation material is a biodegradable and/or selective permeable material.

In one embodiment the separation material is a hydrogel that has a charge, i.e. that contains anionic or cationic groups and has reduced permeability for respectively anionic or cationic species. Further examples and specific embodiments are a polymer functionalized with ((3-acrylamidopropyl)trimethylammonium) that will block Na⁺, a polymer functionalized with 2-acrylamido-2-methyl-1-propanesulfonicacid that will block Cl⁻.

In another embodiment the separation material is polar, for example, a hydrophobic polymer that will block hydrophilic substances. In further embodiments that permeability of the separation material is a function of the crosslink density and/or the porosity of the separation material. As an example, separation material with a high crosslink density acts as a filter with a lower molecular cut-off and thus may be utilized as a selective permeable material for substances with a higher molecular weight. Suitable biodegradable materials are for example described by Gunatillake P and Adhikari R, European Cells and Materials (5) 2003, 1-16, the whole content of which is herein incorporated by reference. Among these are Poly(glycolic acid) (PGA), poly(lactic acid) (PLA) and their copolymers and derivatives, like Poly(d,l-lactic-co-glycolic acid), Polylactones like Poly(caprolactone) (PCL) and their derivatives, Poly(propylene fumarate) (PPF) and its derivatives, Polyanhydrides like Poly [1,6-bis(carboxyphenoxy)hexane], Tyrosine-derived polycarbonates and their derivatives, Polyorthoesters (POE) and their derivatives, Polyurethanes (PU) with non-toxic degradation products like lysine diisocyanate (LDI, 2,6-diisocyanatohexanoate) and other aliphatic diisocyanates like hexamethylene diisocyanate (HDI) and 1,4-butanediisocyanate, e.g. Poly(glycolide-co-γ-caprolactone), Polyphosphazenes like Ethylglycinate Polyphosphazene and their derivatives, and so forth.

Other biodegradable polymers comprise poly(maleic acid), poly(p-dioxanone), poly(trimethylen-carbonate), poly(3-hydroxibutarate), poly(3-hydroxyvalorate and their copolymers. A class of suitable responsive biodegradeable polymers is Poly(N-(2-hydroxypropyl) methacrylamide mono/dilactate), as described by Soga O et al, Biomacromolecules 2004 (5) 818-821, the whole content of which is herein incorporated by reference.

Other suitable biodegradable materials comprise alginate, hyaluronic acid, chitosan, collagen, gelatin, silk or combinations thereof. These biodegradable materials can be used by themselves, or they are being used in a network together with crosslinking agents. The created network will disintegrate after some time, and, given the crosslinking agents have small molecular weights, the latter will be washed out. In another embodiment, the biodegradable material is immobilized in a network that consists of non-biodegradable matter.

The term “hydrogel” as used herein implies that at least a part of the respective material comprises polymers that in water form a water-swollen network and/or a network of polymer chains that are water-soluble. Preferably the hydrogel permeation layer comprises in swollen state 50% water and/or solvent, more preferably 70% and most preferred 80%, whereby preferred solvents include organic solvents, preferably organic polar solvents and most preferred alkanols such as ethanol, methanol and/or (iso-) propanol.

In a first aspect the invention is directed to a nozzle 8 for coaxial extrusion comprising at least two coaxial cylinders, wherein each cylinder contains a cell suspension of a plurality of cells 1, a solution comprising a cell signaling molecule or a separation material 3, wherein cylinders containing a cell suspension or a solution comprising a signaling molecule are separated by a cylinder containing a separation material, and wherein the outermost cylinder contains a separation material.

Such a nozzle can be utilized in order to generate an extruded elongated product comprising a suspension of a plurality of cells and/or a solution comprising a cell signaling molecule enclosed in a layer of separation material. Thus, the nozzle according to the invention allows for the generation of compartments containing a suspension of a plurality of cells and/or a solution comprising a cell signaling molecule surrounded by a layer of separation material. If more than one compartment is generated, these compartments are stacked, i.e. nested.

The outermost cylinder of the nozzle needs to be a cylinder containing separation material and cylinders containing a cell suspension or a solution comprising a signaling molecule are separated by a cylinder containing a separation material. In this way, cylinders containing a liquid such as the cell suspension or the solution comprising a signaling molecule are always separated by a cylinder containing a separation material. Thus, if the contents of the cylinders are extruded according to a method of the present invention no mixing of the liquids will occur.

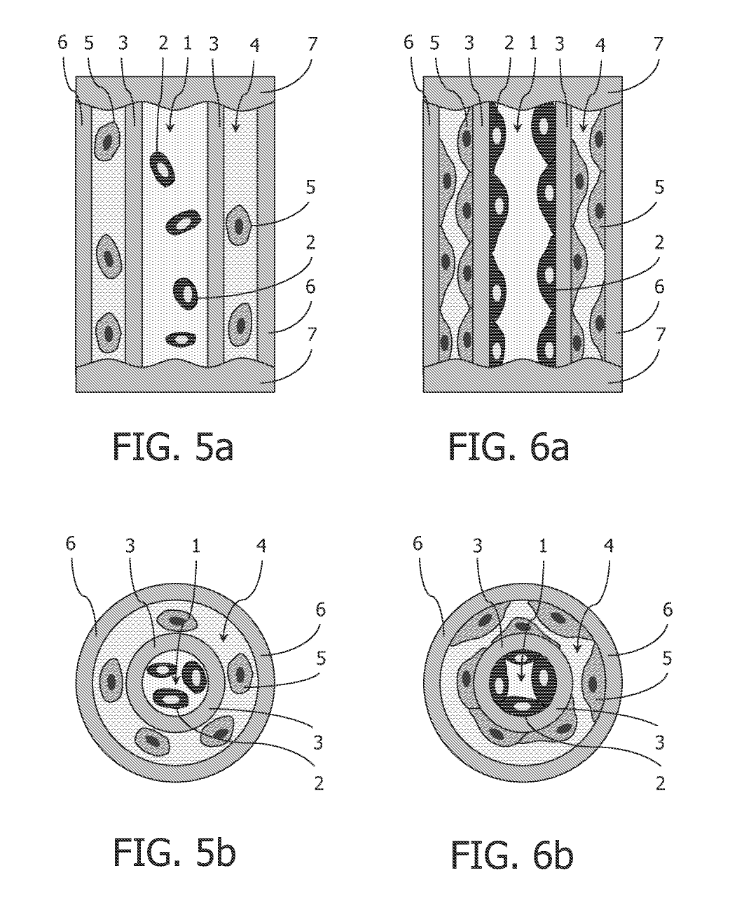

The resulting compartments can be nested/stacked, as for example exemplified by FIGS. 5 and 6. Thus, in one example and preferred embodiment, a compartment containing a suspension with cells of type 1 and a compartment containing a suspension with cells of type 2 can be extruded adjacent to another and the (liquid) contents of the compartments are separated by co-extrusion of a layer of separation material between them. In the resulting extruded product the cells of type 1 are separated from the cells of type 2 by a layer of separation material.

In another example and preferred embodiment a compartment containing a suspension with cells and a compartment containing a solution comprising a cell signaling molecule can be extruded adjacent to another. Thus, in the resulting extruded product the cell suspension is separated from the solution comprising a cell signaling molecule by a layer of separation material.

In a specific embodiment at least one of the cylinders contains a cell suspension. Preferably, the cells are selected from the group consisting of stem cells, progenitor cells, fully-differentiated cells, neural cells, glial cells, Schwann cells, oligodendrocytes, adipose-derived stem cells (ASCs), mesenchymal stem cells (MSCs), Pancreatic progenitor cells and endothelial progenitor cells (EPC).

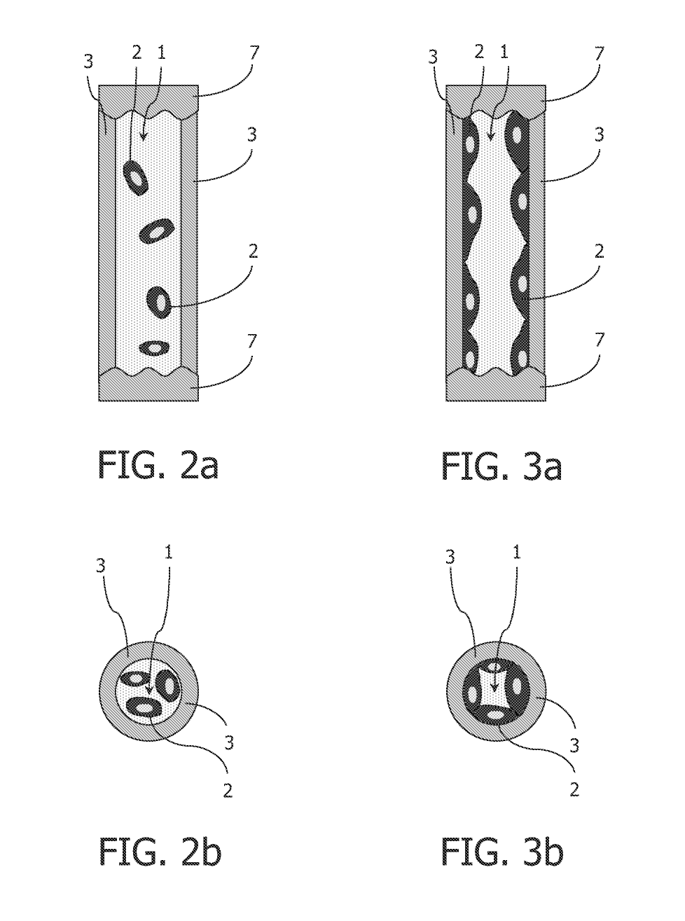

In one embodiment of the invention, as depicted in FIG. 1, the nozzle 8 comprises two coaxial cylinders, wherein the inner cylinder contains a cell suspension 1 comprising a plurality of cells 2 and the outer cylinder contains a separation material 3. The extruded product consists of an elongated compartment containing a cell suspension comprising a plurality of cells which is encapsulated by a layer of separation material. This embodiment of the invention can thus be utilized to produce a single compartment containing a suspension comprising a plurality of cells with a coaxial encapsulation as depicted in FIGS. 1, 2a and 2b. By means of culturing, which depend on the type of cells encapsulated in the compartment and known to the skilled person, these compartments can be transformed into hollow fibrous microtissues as depicted in FIGS. 3a and 3b.

In another embodiment of the invention the nozzle comprises three coaxial cylinders, wherein the inner and the outer cylinders contain a separation material and the middle cylinder contains a cell suspension. Thus, a separation material is extruded in the central cylinder, surrounded by a cell suspension which itself is again encapsulated by an encapsulation layer made of separation material. The resulting extruded product consists of a single elongated compartment comprising a cell suspension which is encapsulated by a layer of separation material on the outside and furthermore comprising a central tube of separation material. As exemplified by FIGS. 1 and 2, this embodiment of the invention can be used to produce a single fibrous compartment containing a suspension 1 comprising a plurality of cells 2 that is encapsulated in a coaxial protective layer made up of the separation material 3.

After an additional culturing step, hollow fibrous microtissue can be obtained (see FIG. 3).

In another embodiment of the invention the nozzle comprises four coaxial cylinders, wherein a first cylinder contains a cell suspension 1 and a second cylinder contains a cell suspension 4 or a solution comprising a cell signaling molecule. The cylinders containing a cell suspension or a solution comprising a cell signaling molecule are separated from each other by a cylinder containing a separating solution and the outermost cylinder likewise contains a separating solution.

Thus, the extruded product consists of one compartment containing a cell suspension and a further compartment containing cell suspension or a solution comprising a cell signaling molecule, wherein these compartments are encapsulated by a layer of separation material and are stacked. In this way, a mixing of the contents contained in the respective compartments will not occur if the separation material is completely impermeable for any of said two contents.

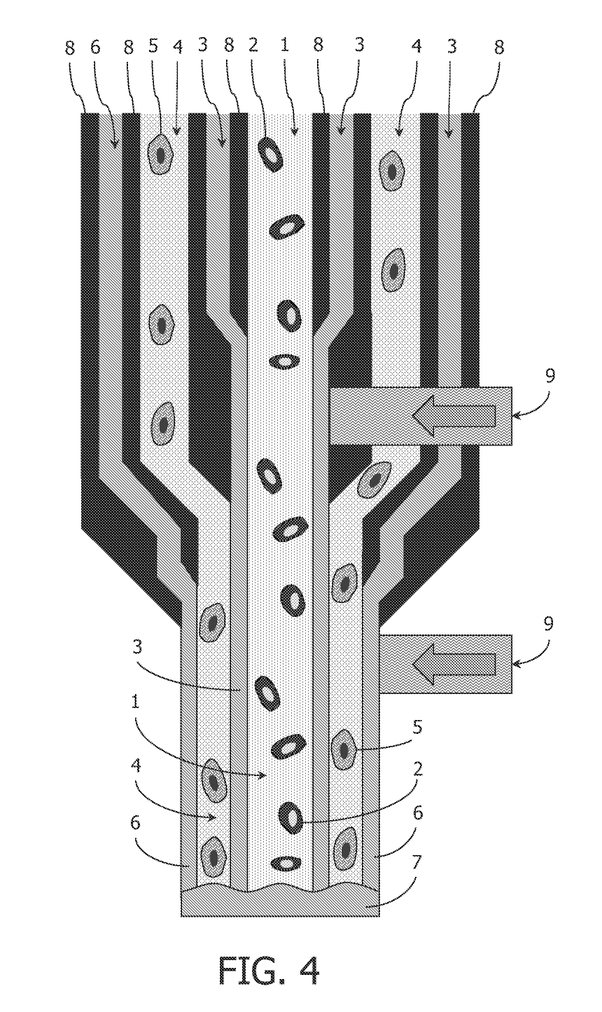

In a specific embodiment, depicted in FIG. 4, the innermost cylinder contains a cell suspension 1 comprising cells of a type 1 (2) which is enclosed by a cylinder containing a separating solution 3 which, in turn, is enclosed by a cylinder containing a cell suspension 4 comprising cells of a type 2 (5). Again, the outermost cylinder contains a separating solution 6. Using this nozzle, fibrous heterogenous tissue such as coaxial microtissue can be engineered as depicted in FIGS. 5 and 6 (showing an early and a further developed state after culturing, respectively).

In a specific embodiment, the cells of type 1 are neuronal cells and the cells of type 2 are glial cells (either Schwann cells or oligodendrocytes). These glial cells can then form the myelin sheath, an insulator for the electrically active neurons which are developing in the inner compartment. As a result, the present invention provides for the generation of functional nerve fibers with a surrounding insulating myelin sheath.

In a further specific embodiment the cells of type 1 are stem cells and the cells of type 2 are feeder cells. In a preferred embodiment one or both of the cell types secret a signaling molecule that is effective on the other cell type. For example, if one of the two cell types are feeder cells these will secrete signaling molecule necessary for the maintenance and/or growth of the other type of cells, which may be stem cells.

In a further specific embodiment, only one of the four cylinders of the nozzle contains a cell suspension, while another one contains a solution comprising a cell signaling molecule. In one embodiment the cell suspension is contained by the innermost cylinder, in another embodiment the solution comprising a cell signaling molecule is comprised by the innermost cylinder. Thus, using this nozzle, the extruded product will consist of a compartment encapsulating a solution comprising a signaling molecule and a compartment encapsulating a cell suspension, wherein both compartments are separated from each other by the encapsulating layer of the separation material.

In a further embodiment, the separation material is biodegradable and/or permeable for the signaling molecule (and not the cells) allowing for the controlled delivery of the signaling molecule to the cells.

In a further specific embodiment the cells are selected from the group consisting of stem cells, progenitor cells, fully-differentiated cells, neural cells, glial cells, Schwann cells, oligodendrocytes, adipose-derived stem cells (ASCs), mesenchymal stem cells (MSCs), Pancreatic progenitor cells, endothelial progenitor cells (EPC), feeder cells and/or cultured cells.

In a further embodiment the signaling molecules are growth factors, cytokines or chemokines. In specific embodiments of the present invention the cell signaling molecule is selected from the group consisting of G-CSF, GM-CSF, SCF, IL-3, IL-6, IGF-1, fibroblast growth factor (FGF), basic FGF (bFGF), transforming growth factor β1 (TGF-β1), activin-A, bone morphogenic protein 4(BMP-4), hepatocyte growth factor (HGF), epidermal growth factor (EGF), β nerve growth factor (β-NGF), retinoic acid, FGF-1, FGF-2, Thrombopoietin (TPO) and/or Erythropoietin (EPO).

As described above, the separating material can (initially) be a completely impermeable material that prevents the encapsulated cell suspension and/or solution comprising a signaling molecule from leaving the compartment. According to a further embodiment the separating material contained in at least one of the cylinders is biodegradable material and/or permeable for a signaling molecule. In a preferred embodiment the separating material in all cylinders is biodegradable and/or permeable for a signaling molecule. If the separation material is a biodegradable material the encapsulating layer(s) will—over time—degrade and thus allow the containments of the compartment(s) to be released. Furthermore, and especially relevant for regenerative medicine, particularly to cell therapy and tissue engineering, the encapsulating layer made up of biodegradable separation material will completely disintegrate and not be present in the patient's body. As an example, if an extruded product according to FIG. 2 or FIG. 3 is injected into a patient, the outermost encapsulating and protective layer 3 of the extruded products of the invention will—after a given time—have completely disintegrated, leaving only the cells 2 that will have differentiated, for example, into a tissue by that time.

According to a further embodiment the separation material is a gel, gelates by auto-gelation or polymerizes. Preferably, the polymerization can be achieved by photo-polymerization. Even more preferably, the photo-polymerization is induced by means of UV radiation.

In a further embodiment an apparatus comprising the nozzle of the invention is disclosed. Preferably, the apparatus is an extruder.

In one embodiment of the invention the extruder further comprises at least one radiation source 9 for photo-polymerization, wherein the radiation source for the photo-polymerization is located close to the end of the respective cylinder containing the photo-polymerizable separation material. In a preferred embodiment the extruder comprises more than one radiation source which are located at the respective ands of the cylinders containing the photo-polymerizable separation material (FIG. 4, (9)).

In a further embodiment the radiation source is a source of UV light. In specific embodiments the radiation source is a high intensity collimated UV source with an intensity of ≧0.1 mW/cm² and ≦5 W/cm², ≧1 mW/cm² and ≦1 W/cm², ≧200 mW/cm² and ≦500 mW/cm², or 0.3 W/cm².

In a further specific embodiment the extruder comprises a shutter, preferably a high speed shutter. The shutter may be connected to the radiation source.

In a further embodiment the extruder comprises means for a pulsed extrusion mode. Pulsed extrusion mode is employed to yield a closed extruded product 7. This extrusion mode is based on a special flow profile, i.e. the time-dependent change of the flow rates of the extruded substances. In a specific embodiment, the flow rate of the separation material is initially (e.g. t=0 sec) at a given value (e.g. 0.5 m/min), while the flow rate for the cell suspension or the solution comprising a cell signaling molecule is 0 m/min. Thus, separation material will be extruded from the cylinder surrounding the cylinder containing the cell suspension but no cell suspension will be extruded. The extruded separation material then forms a seal, for example, if the separation material is photo-polymerizable and a UV source is positioned at the exit point of the cylinder containing the separation material. At a later time (e.g. t=0.5 sec) the flow rate of the cell suspension is increased to the flow rate of the separation material (the flow rate of the separation material may also be decreased beforehand). Thus, the cell solution will be coaxially extruded with the separation material which is photo-polymerized upon exiting its respective cylinder. At a further time (e.g. t=1.5 sec) the flow of the cell solution stopped again, while the flow rate of the separation material is kept up or increased; again a seal of separation material will be formed. The result is a compartment comprising a cell solution that is enclosed by a layer of separation material, wherein the compartment is close-ended 7.

In another embodiment this flow profile for the outer and inner cylinder is repeated many times to yield a multitude of close-end compartments filled with cell suspension. Thus, the present invention further provided a method for the generation of a multitude of close-end compartments filled with cell suspension.

In one example of a pulsed extrusion mode the co-extrusion is performed in a pulsated fashion while the shutter in front of the UV-source is continuously opened. The monomer solution in the outer cylinder is extruded from t=0 at a flow rate of 1 m/min. The flow rate from the cell suspension is equal to 0 m/min at that time. At t=0.5 seconds the flow in the outer cylinder is decreased to 0.5 m/min and at the same time the flow in the inner cylinder is increased from 0 to 0.5 m/min. At t=1.5 sec that flow is stopped again and simultaneously the flow in the outer cylinder is restored to 1 m/min. At t=2 sec all flows are set to zero. The result of this profile is a close-end compartment 7.

In particular embodiments the flow rate of the separation material, a cell suspension, and/or a solution comprising a cell signaling molecule is ≧0 m/min and ≦10 m/min, ≧0 m/min and ≦8 m/min, ≧0 m/min and ≦6 m/min, ≧0 m/min and ≦4 m/min, ≧0 m/min and ≦2 m/min, ≧0 m/min and ≦1.5 m/min, ≧0 m/min and ≦1 m/min, or ≧0 m/min and ≦0.5 m/min.

A further aspect of the invention is directed to the use of the nozzle or the extruder of the invention for the generation of at least one elongated compartment containing at least one cell suspension comprising a plurality of cells encapsulated by a layer of separation material (see FIGS. 2 and 3). Preferably, the elongated compartment comprises at least two stacked/nested compartments as depicted, for example, in FIGS. 5 and 6.

A further aspect of the invention is directed to the use of the nozzle or the extruder of the invention for the generation of at least two connected compartment containing at least one cell suspension comprising a plurality of cells encapsulated by a layer of separation material. Connected compartments refer to compartments that are consecutively extruded and which are separated by a closure 7 as described by, inter alia, Example 3. Preferably, each of the at least two connected compartments comprises at least two stacked/nested compartments as depicted, for example, in FIGS. 5 and 6.

As will be obvious for the skilled person from the description above, also a use for the simultaneous manufacturing of more than one such compartment encapsulated by a layer of separation material is encompassed by the present invention. If more than one encapsulated compartment is produced according to the present invention the resulting compartments are nested, i.e. the resulting extruded product consists of a first compartment encapsulated by a layer of separation material which is surrounded by another compartment encapsulated by a layer of separation material. It will be evident to the skilled person that this “nesting” of compartments can be extended to more than two compartments; preferably, 3, 4 or 5 compartments.

In a preferred embodiment the nozzle or extruder of the invention are used to manufacture two nested compartments, wherein one compartment contains a cell suspension and the other compartment either contains a cell suspension of cells of the same or a different type (FIGS. 5 and 6) or contains a solution comprising a cell signaling molecule.

The methods of the invention enable one to generate compartments containing a cell suspension comprising a plurality of cells and/or a solution comprising a cell signaling molecule surrounded by a layer of separation material. The surrounding layer of separation material encapsulating every compartment protects this compartment and, in case of a cell suspension, ensures contact between the encapsulated cells by retaining the cells within the compartment. Furthermore, the encapsulating layer of separation material may separate two adjacent compartments containing a cell suspension or a solution comprising a cell signaling molecule and can be used to prevent or delay (e.g. if a biodegradable and/or selectively permeable material is used) a mixing of the contents of the two compartments. Furthermore, the separating layer can be used to selectively allow the exchange of contents within the two compartments (e.g. if a selectively permeable material is used). In a specific embodiment, the separation material is a biodegradable material.

In one embodiment the present invention provides for a method for the encapsulation of a cell suspension and/or the generation of encapsulated microtissue, wherein at least one cell suspension, at least one separation material and optionally at least one solution comprising a cell signaling molecule are coaxially extruded, wherein cell suspensions or solutions comprising a signaling molecule are separated by a layer of separation material and wherein the resulting outermost layer consist of separation material (i.e. separation material is extruded out of the outermost cylinder of the nozzle).

In particular embodiments the flow rate of the separation material, a cell suspension, and/or a solution comprising a cell signaling molecule is ≧0 m/min and ≦10 m/min, ≧0 m/min and ≦8 m/min, ≧0 m/min and ≦6 m/min, ≧0 m/min and ≦4 m/min, ≧0 m/min and ≦2 m/min, ≧0 m/min and ≦1.5 m/min, ≧0 m/min and ≦1 m/min, or ≧0 m/min and ≦0.5 m/min.

In further specific embodiments the cells are selected from the group consisting of stem cells, progenitor cells, fully-differentiated cells, neural cells, glial cells,

Schwann cells, oligodendrocytes, adipose-derived stem cells (ASCs), mesenchymal stem cells (MSCs), Pancreatic progenitor cells, feeder cells, endothelial progenitor cells (EPC), pluripontent stem cells, somatic stem cells, myeloid stem cells and/or lymphoid stem cells.

In one embodiment the method of the invention provides for the generation of more than one compartment, wherein the compartments are nested / stacked. As an example and more preferred embodiment the method is directed to the generation of a central compartment containing a cell suspension of cells of a type 1 (FIGS. 5 and 6, (1, 2)) surrounded or encapsulated by a layer of separation material FIGS. 5 and 6, (3) which itself is surrounded by a further compartment containing a cell suspension of cells of a type 2 (FIGS. 5 and 6, (4, 5)) that is likewise encapsulated by a layer of separation material (FIGS. 5 and 6, (6)). Thus, in the resulting extruded product the cells of type 1 are separated from the cells of type 2 by a layer of separation material.

The (temporarily) encapsulated cell suspensions of the present invention are of great value for the field of regenerative medicine, most preferably for in vivo cell delivery.

Preferably, the resulting extruded encapsulated cell suspensions (e.g. FIG. 2 and FIG. 5) are cultured in a further step and under appropriate conditions known to the skilled person to form encapsulated microtissue (e.g. FIG. 3 and FIG. 6).

The method of the invention allows for the generation of hollow fibrous microtissues, wherein a single suspension comprising a plurality of cells and a single surrounding separation material are coaxially extruded in a way that the resulting compartment consist of the cell suspension encapsulated in a single layer of separation material as depicted in, for example, FIG. 2. In a further step the extruded product is then cultured under appropriate conditions known to the skilled person and will eventually form a hollow fibrous microtissue as depicted in, for example, FIG. 3.

The method of the invention allows for the generation of hollow fibrous homo- or heterogeneous microtissues, wherein at least one compartment containing a cell suspension and at least a further compartment containing a cell suspension or a solution comprising a signaling molecule are coaxially extruded. An example of this embodiment is depicted in FIGS. 4 and 5. In a further step the extruded product is then cultured under appropriate conditions known to the skilled person and will eventually form a hollow fibrous microtissue as depicted in, for example, FIG. 6.

Specific embodiments of microtissues generated by the method of the invention comprise nervous tissues, vascular tissues, bone tissue, kidney and/or liver tissue.

Such (temporarily) encapsulated homo- and heterogeneous microtissues are of great value for applications in regenerative medicine, most preferably for in vivo microtissue delivery.

In one embodiment the separation material is biodegradable. Therefore, the encapsulation/separation is temporary since the separation material will degrade over time upon, for example, injection of the encapsulated cell suspension into the body of a patient.

In a further embodiment the separation material is selectively permeable. Therefore, a selective exchange between two adjacent compartments can be implemented. As an example and preferred embodiment one compartment may contain stem cells and the adjacent other one may contain feeder cells. If the separation material is selectively permeably for the signaling molecules produced by the feeder cells but not for both types of cells then the stem cells can be kept in close contact to the feeder cells (ensuring maintenance and growth of the stem cells) without mixing of the two cell populations.

In further embodiments the cells are selected from the group consisting of stem cells, progenitor cells, fully-differentiated cells, neural cells, glial cells, Schwann cells, oligodendrocytes, adipose-derived stem cells (ASCs), mesenchymal stem cells (MSCs), Pancreatic progenitor cells, feeder cells, cultured cells and/or endothelial progenitor cells (EPC).

In one embodiment the method of the invention is directed to a manufacturing process for encapsulating a plurality of cells of a cell suspension, wherein a single suspension comprising a plurality of cells and a single surrounding separation material are coaxially extruded. Thus, the resulting compartment consist of the cell suspension encapsulated in a single protective layer of separation material as depicted in, for example, FIG. 2. In preferred embodiment the generated encapsulated cell suspension can be cultured in a further step and under appropriate conditions known to the skilled person to form a microtissue (e.g. FIG. 3).

According to another embodiment a method for the generation of hollow fibrous microtissues is provided, wherein a single suspension comprising a plurality of cells is coaxially extruded between two layers of separation material. Thus, the separation material is extruded in the central cylinder of the nozzle, surrounded by a cell suspension which itself is again encapsulated by a layer of separation material. In a preferred embodiment the extruded product is then cultured in a further step under appropriate conditions known to the skilled person and will eventually form a hollow fibrous microtissue with a central channel. Examples for microtissues that can be generated by this method comprise nervous tissue (e.g. neuronal cells surrounded by myelin sheath forming glial cells), vascular tissue and/or bone tissue.

In another embodiment a method for the generation of fibrous homo- or heterogeneous microtissues is provided, wherein two suspensions comprising a plurality of cells and two separation materials are coaxially extruded. Thus, as exemplarily depicted in FIGS. 4, 5 and 6, a nozzle comprising four coaxial cylinders is utilized, wherein a first cylinder contains a cell suspension 1 and a second cylinder contains a cell suspension 4. The cylinders containing a cell suspension or a solution comprising a cell signaling molecule are separated from each other by a cylinder containing a separating solution and the outermost cylinder likewise contains a separating solution. The resulting extruded product (exemplarily depicted in FIG. 5) thus comprises a central layer consisting of a cell suspension 1 which is encapsulated by separation material 3 which, in turn is encapsulated by another layer consisting of a cell suspension 4 encapsulated by an outermost layer of separation material 6. In a preferred embodiment the extruded product is then cultured in a further step under appropriate conditions known to the skilled person and will eventually form microtissue.

In a specific embodiment, the cells of the two cell suspensions are cells of the same cell type. In a further preferred embodiment the cells of the two cell suspensions are cells of different cell types (FIGS. 4, 5, 6 (2) and (5)). If cells of a different type are utilized heterogeneous tissue is obtained.

In another specific embodiment one or both of the cell types are cells selected from the group consisting of stem cells, progenitor cells, fully-differentiated cells, neural cells, glial cells, Schwann cells, oligodendrocytes, adipose-derived stem cells (ASCs), mesenchymal stem cells (MSCs), Pancreatic progenitor cells, feeder cells and/or endothelial progenitor cells (EPC).

In specific embodiments the microtissues that are generated according to this method are nervous tissues, vascular tissues, bone tissue, kidney and/or liver tissue.

In a further specific embodiment, the separation material used for the two layers (3) and (6) is the same separation material. In another preferred embodiment the separation material used for the two layers 3 and 6 is different separation material.

In a specific embodiment, depicted in FIG. 4, the innermost cylinder contains a cell suspension 1 comprising cells of a type 1 (2) which is enclosed by a cylinder containing a separating solution 3 which, in turn, is enclosed by a cylinder containing a cell suspension 4 comprising cells of a type 2 (5). Again, the outermost cylinder contains a separating solution 6. Using this nozzle fibrous, heterogenous tissue such as coaxial microtissue can be engineered as depicted in FIGS. 5 and 6 (showing an early and a further developed state after culturing, respectively).

In another specific embodiment, the cells of type 1 are neuronal cells and the cells of type 2 are glial cells (either Schwann cells or oligodendrocytes).

In a further specific embodiment the cells of type 1 are stem cells and the cells of type 2 are feeder cells. In a preferred embodiment one or both of the cell types secret a signaling molecule that is effective on the other cell type.

In another specific embodiment the layer of separation material separating the two cell compartments is biodegradable and/or selectively permeable for the signaling molecule.

In another embodiment a method for the generation of fibrous microtissue is provided, wherein one suspension comprising a plurality of cells, one solution comprising a cell signaling molecule and two separation materials are coaxially extruded. Thus, a nozzle comprising four coaxial cylinders is utilized, wherein two cylinders containing a cell suspension and a solution comprising a cell signaling molecule are present which are separated by a cylinder containing a separation material. Again, the outermost cylinder contains a separation material.

In one particular embodiment the cell suspension is extruded as the central compartment surrounded by the compartment containing the solution comprising a cell signaling molecule. Thus, the extruded product comprises a central compartment consisting of a cell suspension encapsulated by a layer of separation material, which itself is surrounded by a compartment consisting of a solution comprising a cell signaling molecule that is encapsulated by a terminal layer of separation material.

In another specific embodiment the solution comprising a cell signaling molecule is extruded as the central compartment surrounded by the compartment containing the cell suspensions. Thus, extruded product comprises a central compartment consisting of a solution comprising of a cell signaling molecule encapsulated by a layer of separation material, which itself is surrounded by a compartment consisting of a cell suspension that is encapsulated by a terminal layer of separation material.

In a preferred embodiment the extruded product is then cultured in a further step under appropriate conditions known to the skilled person to form microtissue.

This embodiment of the present invention allows for the generation of fibrous tissue such as coaxial microtissue, wherein the cells are extruded together with a solution comprising a cell signaling molecule that is effective in, for example, the differentiation of the cells.

As discussed above, the separation material can be a gel-forming material, a gel and/or a polymerizable material. According to another embodiment of the invention at least one of the extruded separation materials is photo-polymerizable and the method further comprises the step of photo-polymerizing said separation material. As depicted in FIG. 1 or FIG. 4, photo-polymerization can be achieved by means of a radiation source 9. Preferably the radiation source is located close to the point of emission of the separation material from out of the nozzle. Therefore, the radiation source can be located in the vicinity (before or after) the point of emission of the respective separation material from out of the nozzle (as exemplarily depicted in FIG. 4). The radiation source makes sure that photo-polymerization of the separation material has advanced to a state that is sufficient so that another layer, e.g. of a cell suspension, can be co-extruded and thus be brought into contact with the layer of separation material preventing a mixing of the two layers.

In a preferred embodiment the source of radiation is a UV source. In a specific embodiment, the source of radiation is a high intensity collimated UV source with an intensity of ≧0.1 mW/cm² and ≦5 W/cm², ≧1 mW/cm² and ≦1 W/cm², ≧200 mW/cm² and ≦500 mW/cm², or 0.3 W/cm². In further specific embodiments, the peak wavelength of the UV source is ≧100 nm and ≦500 nm, ≧200 nm and ≦400 nm, or ≧300 nm and ≦350 nm.

In another particular embodiment, the dose (dose=time*intensity) used for the photo-polymerization is ≧300 mJ/cm² and ≦1500 mJ/cm², ≧500 mJ/cm² and ≦1000 mJ/cm², or ≧600 mJ/cm² and ≦900 mJ/cm². UV sources suitable for the present invention can be obtained, e.g., at Fusion UV Systems, Inc., USA.

According to another preferred embodiment of the invention the extrusion is carried out in a pulsed co-extrusion mode to enable closure of the compartments. As described above, pulsed extrusion mode is employed to yield a closed extruded product 7, wherein the flowrates of cell suspension, solution comprising a cell signaling molecule and/or separation material within the cylinders are not kept constant but changed during extrusion. Employing such flow profiles, a closure of the extruded compartments is achieved.

Another aspect of the present invention is an encapsulated cell suspension or a microtissue that was produced by the methods of the present invention or using the nozzle of the present invention. The present invention offers the possibility to coaxially encapsulate a cell suspension, preferably of cultured cells, in a single compartment surrounded by a protective layer of a separation material. This coaxial encapsulation of a plurality of cells is achieved by the coaxial extrusion according to the present invention and results in an increased vitality of the encapsulated cells since these cells are now in contact with each other. Furthermore, the formation of microtissues from these encapsulated cells likewise takes place more often and more easily as cell-cell contacts are present.

Since the encapsulation of a plurality of (cultured) cells according to the present invention allows for a cell-cell interaction of the encapsulated cells and results in an increased vitality of the cells and a higher chance of the formation of microtissues, the encapsulated cell suspensions and/or microtissues according to the invention can be utilized as a medicament, for example, in various cell therapy applications and/or in vivo microtissue delivery.

In a further aspect the present invention is directed to the encapsulated cell suspensions or microtissues as a medicament. In a preferred embodiment the present invention is directed to the encapsulated cell suspensions or microtissues as a medicament for applications in regenerative medicine. Examples and preferred embodiments of such applications in regenerative medicine are cell therapy applications and/or microtissue delivery applications, the treatment of any kind of lesions due to stroke or ischemia, paraplegia, neurodegenerative disorders/diseases, Alzheimer's or Parkinson disease, or diabetes.

In a further aspect the present invention is directed to the encapsulated cell suspensions or microtissues are delivered to a damaged tissue area. In this embodiment, stem cells (or microtissues generated from such), which help restore the tissue function of the damaged tissue, are preferably used. Such approach is very promising for the treatment of neurodegenerative diseases, like Alzheimer's or Parkinsson's disease, as well as for the repair of necrotic tissues, as results from cardiac stroke, for example.

In a specific embodiment the encapsulated cell suspensions and/or microtissues are used as a medicament for the treatment of diabetes and symptoms associated therewith. In a specific embodiment the medicament comprises new β-cells and/or stem cells that may develop into such cells. In a further specific embodiment the medicament is delivered to the pancreas of a patient suffering from diabetes.

In another specific embodiment the encapsulated cell suspensions and/or microtissues are used as a medicament for the treatment of neurodegenerative diseases, for example, Parkinson disease and symptoms associated therewith. In a specific embodiment the medicament comprises new neuronal cells and/or stem cells that may develop into such cells. In a further specific embodiment the medicament is delivered to the central nervous system, preferably the brain, of a patient suffering from a neurodegenerative disease. In the case of Parkinson Disease, such cells could, for example, restore dopamine production.

In another specific embodiment the encapsulated cell suspensions and/or microtissues are used as a medicament for the treatment of spinal-cord injuries and/or paraplegia. In a specific embodiment the medicament comprises new neuronal cells and/or stem cells that may develop into such cells. In a further specific embodiment the medicament is delivered to the spinal-cord of a patient suffering from a spinal-cord injury and/or paraplegia to restore motor function.

In another specific embodiment the encapsulated cell suspensions and/or microtissues are used as a medicament for the treatment of myocardial infarction and symptoms associated therewith. Myocardial infarction causes a massive loss of cardiomyocytes, leads to the formation of fibrotic tissue and a hypertrophic response in the remaining myocytes. This results in an impaired cardiac function. In a preferred embodiment the encapsulated cell suspensions or microtissues of the invention are delivered to a patient via intramyocardial injection. More preferably this injection is carried out by means of a catheter. In a specific embodiment the medicament comprises new heart muscle cells (cardiomyocytes) and/or stem cells that may develop into such cells.

In a preferred embodiment the encapsulated cell suspensions or microtissues of the invention are delivered to a patient via injection. More preferably this injection is carried out by means of a catheter. Another way of applying the encapsulated cell suspension or microtissue according to the invention is by means of the photonic needle concept. A photonic needle is a needle into which optical fibers are included to allow for optical imaging during the application of the needle, e.g. for purposes of administration of the encapsulated cell suspensions or microtissues of the invention. The optical fibers can also be used to couple in UV light close to the exit site which can be used to polymerize extruded monomers inside the body. Thus, the photonic needle can be designed as the nozzle of the present invention, allowing for extrusion of the encapsulated cell suspensions or microtissues according to the invention within the patient's body.

In another aspect the microtissues according to the invention are used for the formation of tissue in order to overcome one of the main problems in the field of tissue engineering. If tissues with a thickness of more than about 100 μm are to be generated in vitro, one is generally faced with the problem of supplying the cells located within the tissue with, for example, nutrients and oxygen. Therefore, tissues with a thickness of more than about 100 μm are not producible by current means as the innermost cells constantly die off.

This disadvantage is overcome by the present invention in that the microtissues of the present invention can be stacked on top of each other in order to yield a tissue with a thickness of ≧100 μm, preferably ≧1 cm and even more preferably ≧2 cm.

By stacking the microtissues of the present invention no solid body made up of cells is formed but rather a mesh of fibrous and elongated compartments containing a plurality of cells. In between these encapsulated compartments gas exchange and the flow of, for example, medium can easily be accomplished and the cells can be supplied with the necessary nutrients until, for example, a vascular system has formed within the engineered tissue.

The present invention thus provides method for the generation of tissue comprising the step of stacking at least two layers of microtissue according to the present invention as well as a tissue obtainable by this method. In a specific embodiment the engineered tissue is a heterogeneous engineered tissue.

In a further specific embodiment the tissue has a thickness of ≧100 μm and ≦5 cm. In another specific embodiment the tissue has a thickness of ≧300 μm and ≦2 cm. In another specific embodiment the tissue has a thickness of ≧500 μm and ≦1 cm.

In a further specific embodiment ≧1 and ≦100 microtissues are stacked. In a further specific embodiment ≧10 and ≦90, ≧20 and ≦80, ≧30 and ≦70 or ≧40 and ≦60 microtissues are stacked. In further specific embodiments ≧2 and ≦10, ≧3 and ≦9, ≧4 and ≦8, or ≧5 and ≦7 microtissues are stacked.

While the invention has been illustrated and described in detail in the drawings and foregoing description, such illustration and description are to be considered illustrative or exemplary and not restrictive, the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. Any reference signs in the claims should not be construed as limiting the scope.

### EXAMPLES

### Example 1

Eukaryotic epidermoid carcinoma cells A431 (ATCC# CRL-1555) were cultured in culture medium (Dulbecco's Modified Eagle's Medium; DMEM, Gibco), supplemented with 10% fetal calf serum (FCS), 1% penicillin/streptomycin and 1% Glutamax (Invitrogen) at standard cell culturing conditions (37° C., 5% CO2, ˜95% humidity).

After two days the cells formed a confluent cell layer and the medium was removed. The cells were washed twice with phosphate buffered saline (PBS) and subsequently incubated for 10 minutes with EDTA/trypsin. 10 ml of culture medium was added to yield a cell suspension.

The cell suspension (1×10⁵ cells/ml) was fed into the central cylinder of a coaxial extrusion apparatus, while the separation material [an aqueous solution of acrylamide (10 w/w %), biscarylamide (0.5 w/w % and photoinitiator irgacure 2959 (0.1%)] was fed into the outer cylinder.

The monomer solution in the outer cylinder and the cell suspension in the inner cylinder were coextruded at a flow rate of 1 m/min.

A high intensity collimated UV source (0.3 W/cm²) equipped with a high speed shutter was placed at the exit of the extruder's nozzle. During co-extrusion of the separation material and the cell suspension the shutter was opened and closed at a frequency of 1 Hz.

Photopolymerization of the acrylamide/bisacrylamide mixture resulted in the formation of open-ended compartments comprising a plurality of A431 cells and encapsulated by a surrounding layer of polyacrylamide.

### Example 2

Example 1 is repeated employing pulsed extrusion mode, while the shutter in front of the UV-source was opened continuously. The flow profile utilized is as follows:

At t=0, the monomer solution in the outer cylinder is extruded with a flow rate of 1 m/min, while the flow rate of the cell suspension is 0 m/min at that time.

At t=0.5 seconds the flow rate in the outer cylinder is decreased to 0.5 m/min and at the same time the flow in the inner cylinder (cell suspension) is increased from 0 to 0.5 m/min.

At t=1.5 seconds the flow of the cell suspension is stopped again, while the flow in the outer cylinder is restored to 1 m/min simultaneously.

At t=2 sec all flows are set to zero.

This experimental set-up results in the formation of a closed-ended compartment comprising the A431 cell suspension and encapsulated by a surrounding layer of polyacrylamide.

### Example 3

Example 2 is repeated, wherein the flow profile is repeated 30 times.

This experimental set-up results in the formation of approx. 30 connected closed-ended compartments comprising the A431 cell suspension and encapsulated by a surrounding layer of polyacrylamide.

### Example 4

Example 2 is repeated, wherein the nozzle that is utilized comprised three coaxial cylinders.

The monomer solution is fed into the central and into the outer cylinders, while the cell suspension is fed into the intermediate cylinder. Coextrusion is performed with the flow rates given above. Two high intensity collimated UV sources (0.3 W/cm2) equipped with high speed shutters are placed at the exit points of the respective cylinders containing the separation material.

This experimental set-up results in the formation of a closed elongated compartment comprising the A431 cell suspension that is encapsulated by a layer of separation material on the outside and furthermore comprising a central tube of separation material.

### Example 5

The experimental set-up is similar to that described for Example 2. However, the nozzle that is utilized comprises four coaxial cylinders.

Furthermore, the A431 cells are exchanged for primary neural cells that were cultured under conditions known to the skilled artisan. Additionally, a cell suspension comprising a plurality (1×105/ml) of primary glial cells is provided, wherein the glial cells had been cultured under conditions known to the skilled artisan.

The cell suspension comprising the neural cells is fed into the central cylinder. The cell suspension comprising the glial cells is fed into the third cylinder (counting from the innermost cylinder). Separation material is fed into the outermost cylinder and into the cylinder in between the cylinders containing the cell suspensions.

Thus, the cylinders of the extruder are fed in the following manner (starting from the innermost cylinder):

Neural cell suspension-separation material-glial cell suspension-separation material.

Two high intensity collimated UV sources (0.3 W/cm²) equipped with high speed shutters are placed at the exit points of the respective cylinders containing the separation material.

This experimental set-up results in the formation of a closed-ended compartment comprising the cell suspension of neural cells, encapsulated by a surrounding layer of polyacrylamide, surrounded by the cell suspension of glial cells, likewise encapsulated by a surrounding layer of polyacrylamide.

### Example 6

Example 5 is repeated, wherein the A431 cells were exchanged for Human embryonic stem cells (hESCs) and the second cell suspension is exchanged for a solution comprising fibroblast growth factor-20 (FGF-20) at a concentration of 10 ng/ml of medium.

Thus, the cylinders of the extruder are fed in the following manner (starting from the innermost cylinder):

hESCs cell suspension-separation material-FGF-20 solution-separation material.

This experimental set-up results in the formation of a closed-ended compartment comprising the cell suspension of hESCs, encapsulated by a surrounding layer of polyacrylamide, surrounded by the solution comprising the cell signalling molecule FGF-20 and encapsulated by an outer surrounding layer of polyacrylamide.

### Example 7

Example 6 is repeated, wherein the nozzle that is utilized comprised six coaxial cylinders. Furthermore, an additional cell suspension comprising PA6 mouse stromal cells (1×10⁵/ml) is provided.

The cylinders of the extruder are fed in the following manner (starting from the innermost cylinder):

Solution comprising the cell signaling molecule-separation material-hESC cell suspension-separation material-PA6 cell suspension-separation material.

Three high intensity collimated UV sources (0.3 W/cm²) equipped with high speed shutters are placed at the exit points of the respective cylinders containing the separation material.

This experimental set-up results in the formation of a closed-ended compartment comprising a solution of FGF-20 encapsulated by a layer of polyacrylamide, followed by the cell suspension of hESCs encapsulated by a surrounding layer of polyacrylamide, followed by the cell suspension of PA6 cells surrounded by an outer surrounding layer of polyacrylamide.

The resulting extruded product is then cultured for three weeks under conditions known to the skilled artisan. Subsequently, the compartment formerly comprising the hESC suspension is cut open and the generated microtissue is analyzed for the expression of tyrosine hydroxylase. A large number of cells expressing tyrosine hydroxylase are expected to be detected, which indicates that dopaminergic neurons derived from hESCs have been generated. Such dopaminergic neurons will be highly useful for cell therapy in Parkinson's disease.

### Example 8

Examples 1 and 2 are repeated, wherein the separation material is the biodegradable separation material: Polylactic acid-based crosslinkers (Bis[poly(L-lactide)]3,6,9,12,15-pentaoxaheptadecane-1,17-dioxycarbonyl terminated diacrylate)

The resulting generated encapsulated compartments are comparable to those of Examples 1 and 2.

1. Nozzle (8) for coaxial extrusion comprising at least two coaxial cylinders, wherein each cylinder contains a cell suspension (1), a solution comprising a cell signaling molecule or a separation material (3),
wherein cylinders containing a cell suspension or a solution comprising a signaling molecule are separated by a cylinder containing a separation material, and
wherein the outermost cylinder contains a separation material.

2. Nozzle according to claim 1 comprising four coaxial cylinders, wherein a first cylinder contains a cell suspension (1) and a second cylinder contains a cell suspension (4) or a solution comprising a cell signaling molecule.

3. Nozzle according to claim 1, wherein the separating material in at least one of the cylinders is biodegradable material and/or selectively permeable for a signaling molecule.

4. Extruder comprising the nozzle according to claim 1.

5. Use of the nozzle according to claim 1 or the extruder according to claim 4 for the generation of at least one compartment containing a cell suspension encapsulated by a layer of separation material.

6. Method for the encapsulation of a cell suspension and/or the generation of microtissue the method comprising:
coaxially extruding at least one cell suspension, at least one separation material and optionally at least one solution comprising a cell signaling molecule,
wherein cell suspensions or solutions comprising a signaling molecule are separated by separation material and
wherein the resulting outermost layer consist of separation material.

7. Method according to claim 6 for the generation of an encapsulated cell suspension, wherein one suspension comprising a plurality of cells and one surrounding separation material are coaxially extruded.

8. Method according to claim 6 for the generation of microtissue, wherein two suspensions comprising a plurality of cells and two separation materials are coaxially extruded.

9. Method according to claim 6 for the generation of microtissue, wherein one suspension comprising a plurality of cells, one solution comprising a cell signaling molecule and two separation materials are coaxially extruded.

10. Method according to claim 6, wherein at least one separation material is photo-polymerizable and the method further comprises the step of photo-polymerizing the at least one separation material.

11. An encapsulated cell suspension or microtissue obtainable by the method according to claim 6.

12. The encapsulated cell suspension or microtissue according to claim 11 as a medicament.

13. The encapsulated cell suspension or microtissue according to claim 11 as a medicament for cell therapy and/or microtissue delivery applications.

14. Method for the generation of tissue comprising the step of stacking at least two layers of microtissue according to claim 11.

15. A tissue comprising at least two stacked layers of microtissue according to claim 11.
